(19) United States
(12) Patent Application Publication
(10) Pub. No.: US 2008/0261924 A1
(43) Pub. Date: Oct. 23, 2008

(54) METHOD FOR THE TREATMENT OF BONE DISORDERS

(76) Inventors: Pamela Jean Schofield, Cincinnati, OH (US); Henry Van den Berg, Cincinnati, OH (US); David Ernest Burgio, Liberty Township, OH (US); Arkadi Aaron Chines, Cincinnati, OH (US)

Correspondence Address: HOFFMANN-LA ROCHE INC.;PATENT LAW DEPARTMENT, 340 KINGSLAND STREET, NUTLEY, NJ, 07110, US

(21) Appl. No.: 12/163,155

(22) Filed: Jun. 27, 2008

Related U.S. Application Data
(63) Continuation of application No. 11/725,896, filed on Mar. 20, 2007.
Application No. 11/725,896 is a continuation of application No. 10/321,208, filed on Dec. 17, 2002, now abandoned.
(60) Provisional application No. 60/344,875, filed on Dec. 21, 2001.

(51) Int. Cl. A61K 31/662; A61P 19/00

(52) U.S. Cl. 514/108

(57) ABSTRACT

Disclosed are methods for treating bone disorders in mammals. The methods comprise a loading period with a bisphosphonate followed by a maintenance period. The loading dose is two to twenty times per day greater than the corresponding maintenance dose. Also disclosed are compositions and kits for implementing the methods disclosed herein.

### FIELD OF THE INVENTION

This invention relates to methods of increasing bone mass and reduction of fractures for the treatment of osteoporosis and other bone metabolic disorders. In particular, this invention relates to such methods of treatment by the administration of a loading dose of a bone-active phosphonate followed by a maintenance dosing regimen.

### BACKGROUND OF THE INVENTION

The most common metabolic bone disorder is osteoporosis. Osteoporosis can be generally defined as the reduction in the quantity of bone, or the atrophy of skeletal tissue due to an imbalance in the normal resorption/formation cycle of bone within the bone remodeling unit. In general, there are two types of osteoporosis: Primary and secondary. Secondary osteoporosis is the result of an identifiable disease process or agent. For example, glucocorticoid steroids are known to induce osteoporosis. See, for example American College of Rheumatology Ad Hoc Committee on Glucocorticoid-Induced Osteoporosis, “Recommendations for the Prevention and Treatment of Gluococorticoid-Induced Osteoporosis”, Arthritis & Rheumatism, Vol. 44, No. 7, July 2001, pg 1496-1503 © 2001; B. P. Lukert, M.D., F.A.C.P. “Glucocorticoid-Induced Osteoporosis”, Primer in the Metabolic Bone Diseases and Disorders of Mineral Metabolism, Fourth Edition, Chapter 55, pgs 292-296, Publication of the American Society for Bone and Mineral Research, Murray J. Favus, M.D. Editor, Dept of Medicine, The University of Chicago Medical Center, Chicago, Ill. Approximately 85% of all osteoporosis is primary osteoporosis. See for example, Marjorie M. Luckey, M.D., “Evaluation of Postmenopausal Osteoporosis”, Primer on the Metabolic Bone Diseases and Disorders of Mineral Metabolism, 4^{th} Edition, pgs 273-277, Murray J. Favus, M.D. Editor, Dept of Medicine, The University of Chicago Medical Center, Chicago, Ill.; and “Osteoporosis Prevention, Diagnosis, and Therapy” JAMA, Feb. 14, 2001-Vol. 285, No. 6; pgs 785-795. Such primary osteoporosis includes postmenopausal osteoporosis, age-associated osteoporosis (affecting a majority of individuals over the age of 70 to 80) and idiopathic osteoporosis.

For some osteoporotic individuals the loss of bone tissue is sufficiently great so as to cause mechanical failure of the bone structure. Bone fractures often occur, for example, in the hip and spine of women suffering from postmenopausal osteoporosis. Kyphosis (abnormally increased curvature of the thoracic spine) may also result. Although its etiology is not fully understood, there are many risk factors thought to be associated with osteoporosis. These include low body weight, low calcium intake, physical inactivity, and estrogen deficiency.

Many compositions and methods are described for the “treatment” of osteoporosis. Many of these include the use of bisphosphonates or other bone-active phosphonates. See, for examples, J. Y. Reginster, et al., “Randomized Trial of the Effects of Risedronate on Vertebral Fractures in Women with Established Postmenopausal Osteoporosis”, Osteoporosis International, (2000) 11: pgs 83-91; Steven T. Harris, MD, et al., “Effects of Risedronate Treatment of Vertebral and Nonvertebral Fractures in Women With Postmenopausal Osteoporosis, A Randomized controlled Trial” Journal of the American Medical Association, Oct. 13, 1999, Vol. 282, No. 14, pgs 1344-1352.

Continuous and cyclic administration of bisphosphonates alone or with other medicants such as parathyroid hormone, calcium and vitamin D have also been suggested as a therapy for osteoporosis. See, for example American College of Rheumatology Ad Hoc Committee on Glucocorticoid-Induced Osteoporosis, “Recommendations for the Prevention and Treatment of Gluococorticoid-Induced Osteoporosis”, Arthritis & Rheumatism, Vol. 44, No. 7, July 2001, pg 1496-1503 © 2001; J. Y. Reginster, et al., “Randomized Trial of the Effects of Risedronate on Vertebral Fractures in Women with Established Postmenopausal Osteoporosis”, Osteoporosis International, (2000) 11: pgs 83-91; Steven T. Harris, MD, et al., “Effects of Risedronate Treatment of Vertebral and Nonvertebral Fractures in Women With Postmenopausal Osteoporosis, A Randomized controlled Trial” Journal of the American Medical Association, Oct. 13, 1999, Vol. 282, No. 14, pgs 1344-1352.

Applicants have found, surprisingly, that the administration of a high dose of a bone-active phosphonate followed by a lower maintenance dose decreases bone turnover and increases bone mass at a faster rate leading to faster fracture reduction. This may be particularly useful in patients who experience high bone turnover, such as cancer and transplant patients.

### SUMMARY OF THE INVENTION

The present invention provides methods of increasing bone mass and/or reducing fractures in a subject afflicted with bone loss. The method comprises the steps of: (a) administering a loading dose of a bisphosphonate for a period of time of from about 7 to about 180 days, more preferably from about 14 to about 60 days, followed by (b) administering a continuous maintenance dose of a bisphosphonate. The loading dose comprises a level of bisphosphonate of from about 2 to about 20 times, preferably from about 3 times to about 10 times, more preferably from about 3 times to about 6 times greater than the corresponding maintenance dose. The loading dose is administered over a period of time from about 7 to about 180 days. For oral administration, the loading dose is administered every day or ever other day whereas the maintenance dose may be administered every day, twice a week, weekly, bi-weekly, or monthly.

### DETAILED DESCRIPTION OF THE INVENTION

The methods of the present invention comprise the administration of a loading dose of a bone-active phosphonate and a maintenance dose of a bone-active phosphonate. Specific compounds and compositions to be used in these processes must, accordingly, be pharmaceutically-acceptable.

### Definitions:

“Administering”, as used herein means any method which, in sound medical practice, delivers the actives used in this invention to the subject treated in such a manner so as to be effective in the building of bone. The actives may be administered by any of a variety of known methods of administration, e.g., orally, dermatomucosally (for example, dermally, sublingually, intranasally, and rectally), parenterally (e.g., subcutaneous injection, intramuscular injection, intra-articular injection, intravenous injection), topically (transdermal) and inhalating. Thus, specific modes of administration include, but are not limited to, oral, transdermal, mucosal, sublingual, intramuscular, intravenous, intrapertioneal, subcutaneous administration, and other topical application.

“Loading dose”, as used herein, means the dose initially administered to a patient. The dose is an effective amount to achieve the desired results.

“Loading period”, as used herein means the period of time in which the initial dose is administered to a subject.

“Maintenance dose”, as used herein, means the dose administered to a subject following the loading period. The dose is an effective amount to achieve the desired results.

“Maintenance period”, as used herein means, means the period of time following the loading period in which the subject is continuously administered a dose of bisphosphonate at a dosage level lower than the loading dose.

“Safe and effective amount”, as used herein means an amount large enough to significantly induce a positive modification in the symptoms and/or condition to be treated in a subject, but small enough to avoid serious adverse side effects, commensurate with a reasonable benefit/risk ratio. The safe and effective amount will vary with such factors as the particular condition being treated, the age and physical condition of the patient, the duration of treatment, the nature of concurrent therapy, the specific dosage form to be used, and the dosage regimen employed.

### Method:

The method of the present invention comprises the steps of
(a) administering a loading dose of a bisphosphonate for about 7 days to about 180 days of a bisphosphonate; and
(b) administering after step (a) a continuous maintenance dose of a bisphosphonate. wherein said loading dose is from about two (2) times to about twenty (20) times greater than said maintenance dose.

Accordingly, the loading dose period is comprised of a separate administration regimen for the bisphosphonate. The bisphosphonate must be given with sufficient frequency in the loading dose period in order to achieve the physiological effect in the subject being treated. For example, an oral dosage unit of bisphosphonate is preferably administered every day of the loading period. It may be desirable to administer one type of bisphosphonate on some treatment days and another type on another treatment day.

In addition, a bisphosphonate must be given at least once every three months after the loading period. However, a bisphosphonate may be given every day, every other day, twice a week, weekly, bi-weekly, once a month or every other month. It may be desirable to administer one type of bisphosphonate on some treatment days, and another type on another treatment day.

The specific period of time and the frequency of dosing which is sufficient to achieve an increase in the net skeletal mass of the subject may depend on a variety of factors. Such factors include, for example, the specific actives employed, the amount of actives administered, the mode of administration (i.e. oral or parenteral) the age and sex of the subject, the specific disorder to be treated, concomitant therapies employed, the general physical health of the subject, the extent of bone loss in the individual, and the nutritional habits of the individual.

The therapeutic regimen utilizing the methods of this invention are preferably continued for at least about twenty four months. Of course, a therapeutic regimen may be continued indefinitely, according to sound medical practice.

A preferred method for the treatment of a bone disorder includes an initial diagnostic step, to determine the presence of the disorder. Thus, a preferred method of this invention comprises the steps of performing a diagnostic on a subject for the detection of high bone turnover. High bone turnover can be defined when the net bone turnover is elevated and bone resorption is greater than bone formation. Upon obtaining a positive result from said diagnostic, administering the actives according to the methods of this invention is then implemented. Measurement of biochemical markers may be used to determine the rate of bone turnover. Bone remodeling may be confirmed by histomorphology.

Suitable diagnostics for the detection of established osteoporosis are also well known in the art. Such methods include the measurement of the radiodenisty of skeletal radiographs, quantitative computerized tomography, single energy photon absorptiometry, and dual-energy photon absorptimoetry. Diagnostic techniques among those useful herein are described in W. A. Peck et al., Physician's Resource Manual on osteoporosis (1987), published by the National Osteoporosis Foundation (incorporated by reference herein).

The bone-active phosphonate (bisphosphonate, diphosphonate), as used herein encompasses acid, salts, and derivatives thereof. Nonlimiting examples of bisphosphonates useful herein include the following: 1-hydroxy-2-(3-pyridinyl)-ethylidene-1,1-bisphosphonic acid (risedronate) as described in U.S. Pat. No. 5,583,122, to Benedict, et al., Dec. 10, 1996, which is incorporated by reference herein in its entirety. 4-amino-1-hydroxybutylidene-1,1-bisphosphonic acid (alendronic acid) as described in U.S. Pat. No. 4,621,077, to Rosini et al., Nov. 4, 1986; U.S. Pat. No. 4,922,007, to Kieczykowski et al., May 1, 1990 and U.S. Pat. No. 5,019,651, to Kieczykowski, May 28, 1991, all of which are incorporated by reference herein in their entirey. 3-amino-1-hydroxypropylidene-1,1-bisphosphonic acid (pamidronate) (4-cblorophenyl)thiomethan-1,1diphosphonic acid (tiludronate) as described in U.S. Pat. No. 4,876,248 to Breliere et al., Oct. 24, 1989, which is incorporated by reference herein in its entirety. 1.1-dicloromethylene-1-1-diphosphonic acid (clodronate) ad described in Belgium Patent 672,205 (1966) which is incorporated by reference herein in its entirety. Cyclohepylaminomethylene-1,1-bisphospnonic acid (cimadronate), as described in U.S. Pat. No. 4,970,335, to Isomura et al., Nov. 13, 1990 which is incorporated herein by reference in its entirety. 1-hydroxy-3-(N-methyl-N-pentylamino)propylidene-1,1-bisphosphonic acid (ibandronate) which is described in U.S. Pat. No. 4,927,814, May 22, 1990, which is incorporated by reference herein in its entirety. 1-hydroxy-2-(1H-imidazol-1-yl)ethylidene-1,1-bisphosphonic acid (zolendronate).

Preferred bisphosphonates are selected from the group consisting of risedronate, ibandronate, pamidronate, alendronate, cimadronate, tiludronate, zolendronate, clodronate, piridronate, pharmaceutically-acceptable salts thereof and mixtures thereof.

The amount of bisphosphonate to be administered depends upon its potency. The potency of a particular bisphosphonate can be expressed in terms of its “LED” or “least effective dose”, which is a minimum dose of bisphosphonate expressed in mg P/kg (milligrams phosphorus in the compound per kilogram weight of the subject) that is effective, by itself, to cause a significant inhibition of bone resorption. The specific LEDs of the bisphosphonates will vary depending upon their chemical composition, and their method of administration (i.e., oral or parenteral). The lower the LED, the more potent the bisphosphonate and, generally, it is desirable to administer the high potency bisphosphonate in lower doses and on a fewer number of days. Likewise, the higher the LED, the less potent the bisphosphonate, and generally, it is desirable to administer the low potency bisphosphonate in higher doses and on a greater number of days. The LEDs for oral dosing would be higher, depending upon the systemic absorption of the phosphonate. Typically, absorption from oral administration is from about 1% to about 10%. Thus, oral LEDs are typically about ten- to about one hundred-fold higher than the parenteral LEDs.

There are a number of models that can be used to determine the LEDs for the bone-active phosphonates. These are further described in U.S. Pat. No. 4,761,406, Flora et al., Aug. 2, 1988 which is incorporated herein in its entirety be reference.

### Dosage Forms:

The bone-active phosphonate may be administered in any of a variety of pharmaceutically-acceptable compositions. Such compositions may comprise an active and a pharmaceutically-acceptable carrier. Pharmaceutically-acceptable carriers include solid or liquid filler diluents or encapsulating substances, and mixtures thereof, that are suitable for administration to a human or lower animal. The term “compatible,” as used herein, means that the components of the pharmaceutical composition are capable of being commingled with the actives, and with each other, in a manner such that there is no interaction which would substantially reduce the pharmaceutical efficacy of the pharmaceutical composition under ordinary use situations. Pharmaceutically-acceptable carriers must, of course, be of sufficiently high purity and sufficiently low toxicity to render them suitable for administration to the subject being treated.

Some examples of the substances which can serve as pharmaceutical carriers are: sugars, such as lactose, glucose and sucrose; starches, such as corn starch and potato starch; cellulose and its derivatives, such as sodium carboxymethylcellulose, ethylcellulose, cellulose acetate; powdered tragacanth; malt; gelatin; talc; stearic acid; magnesium stearate; vegetable oils, such as peanut oil, cottonseed oil, sesame oil, olive oil, corn oil and oil of theobroma; ppolyols such as propylene glycol, glycerin, sorbitol, mannitol, and polyethylene glycol; agar; alginic acid; pyrogen-free water; isotonic saline; phosphate buffer solutions; wetting agents and lubricants such as sodium lauryl sulfate; coloring agents; flavoring agents; and preservatives. Other compatible pharmaceutical additives and actives may be included in the pharmaceutically-acceptable carrier for use in the compositions of the present invention.

The choice of a pharmaceutically-acceptable carrier to be used in conjunction with the active is determined by the way the active is to administered. If the active is to be injected, the preferred pharmaceutical carrier is sterile water, physiological saline, or mixtures thereof. The pH of such parenteral composition is preferably adjusted to about 7.4. Suitable pharmaceutically-acceptable carriers for topical application include those known in the art for use in creams, gels, tapes, patches, and similar topical delivery means.

The pharmaceutically-acceptable carrier employed in conjunction with the actives is used at a conscentration sufficient to provide a practical size to dosage relationship. The pharmaceutically-acceptable carriers, in total, may comprise from about 0.1% to about 99.9% by weight of the pharmaceutical compositions of the present invention, preferably from about 5% to about 80% and most preferably from about 10% to about 50%.

A preferred method of administering bisphosphonates is orally, in a unit-dosage form (i.e., a dosage form containing an amount of active suitable for administration in one single dose, according to sound medical practice). Preferred unit dosage forms for bisphosphonate include tablets, capsules, suspensions, and solutions, comprising a safe and effective amount of active. Pharmaceutically-acceptable carriers suitable for the preparation of unit dosage forms for oral administration are well known in the art. Their selection will depend on secondary considerations like taste, cost, shelf stability, which are not critical for the purposes of the present invention, and can be made without difficulty by a person skilled in the art. Preferably, oral unit dosage forms of risedronate for the loading dose comprise from about 15 mg to about 50 mg per day, more preferably from about 20 mg to about 40 mg per day and most preferably from about 25 mg to about 35 mg per day. The oral unit dosage forms of the bone-active phosphonate for the maintenance dose preferably contains from about 2.5 to about 15 mg per day from about 5 to about 10. For alendronate the loading dose comprises from about 15 mg to about 70 mg per day. More preferably from about 20 mg to about 50 mg per day, and most preferably about 25 mg to about 40 mg per day. Equivalent doses can be given every other day, twice a week, weekly, bi-weekly or monthly.

Another preferred method of administering bisphosphonates is subcutaneous injection in a unit dosage form. Preferred unit dosage forms for injectable bone active bisphosphonate include sterile solutions of water, physiological saline, or mixtures thereof. The pH of said solutions should be adjusted to about 7.4. Preferably, unit dosage forms of risedronate for the loading dose comprise from about from about 0.75 mg to about 15.0 mg per month and more preferably from about 1.5 mg to about 10 mg per month. The unit dosage forms of the bone-active phosphonate for the maintenance dose preferably contains from about 0.75 mg to about 6 mg per month and more preferably from about 1.5 mg to about 3 mg per month. Equivalent dosage amounts may be given every two weeks, every month, every other month or every three months.

### Kits:

This invention also provides kits for conveniently and effectively implementing the methods of this invention. Such kits comprise one or more unit doses of bone-active phosphonate for the loading period, one or more unit doses of bone-active phosphonate for the maintenance period, and a means for facilitating compliance with methods of this invention. Such kits provide a convenient and effective means for assuring that the subject to be treated takes the appropriate active in the correct dosage in the correct manner. The compliance means of such kits includes any means which facilitates administering the actives according to a method of this invention. Such compliance means includes, instructions, packaging, and dispensing means, and combinations thereof. Examples of packaging and dispensing means are well known in the art, including those described in U.S. Pat. No. 4,761,406, Flora et al., issued Aug. 2, 1988; and U.S. Pat. No. 4,812,311, Uchtman, issued Mar. 14, 1989, all incorporated by reference herein.

The following non-limiting examples illustrate the compositions, process and uses of the present invention.

### EXAMPLE 1

A female patient weighing approximately 60 kg and diagnosed with postmenopausal osteoporosis is treated by a method of this invention. Specifically for thirty days the patient is given 30 mg per day of risedronate orally. Immediately following, the patient is given 35 mg per week of risedronate orally for two years. A biopsy of iliac crest bone is taken at two years and reveals an increase in mean wall thickness of the remodeling units compared to her baseline biopsy.

### EXAMPLE 2

A male weighing approximately 70 kg diagnosed with prostrate cancer and high bone turnover is treated by a method of this invention. Specifically each day for fourteen days the patient takes 35 mg of alendronate per day. At the end of the period, the patient then takes a maintenance dose of 70 mg per week of alendronate orally for one year.

### EXAMPLE 3

A female weighing about 58 kg is diagnosed with glucocorticoid-induced osteoporosis. The subject is then treated by a method of this invention. Specifically the subject is given 30 mg risedronate orally per day for a period of 30 days. At that time, the dose is switched to the maintenance dose of 35 mg orally every two weeks for three years.

### EXAMPLE 4

A male patient weighting approximately 67 kg is given intravenously; a total of 9 mg divided equally into two weekly doses (4.5 mg per week on days 1 and 8) of risedronate. The maintenance dose of 3 mg given on Day 29 (after first loading dose) followed by 3 mg every other month from Day 29.

While particular embodiments of the subject invention have been described, it will be obvious to those skilled in the arts that various changes and modification of the subject invention can be made without departing from the spirit and scope of the invention. It is intended to cover, in the appended claims all such modifications that are within the scope of this invention.

What is claimed is:

1. A method of increasing bone mass in a human or other animal with high bone turnover comprising the steps of
(a) administering a loading dose for about 7 days to about 180 days of a bisphosphonate; and
(b) administering after step (a) a continuous maintenance dose of a bisphosphonate, wherein the loading dose is from about two times to about twenty times greater than the maintenance dose.

2. The method of claim 1 wherein the bisphosphonate of step (a) is selected from the group consisting of risedronate, ibandronate, pamidronate, alendronate, cimadronate, tiludronate, zolendronate, clodronate, piridronate, pharmaceutically-acceptable salts thereof and mixtures thereof.

3. The method of claim 2 wherein the bisphosphonate is administered orally, dermatomucosally, parenterally, transdermally or inhaling.

4. The method of claim 3 wherein the bisphosphosphonate is administered orally, transdermally, intramuscularly, intravenously and subcutaneously.

5. The method of claim 4 wherein the bisphosphonate of step (a) is administered orally.

6. The method of claim 5 wherein the bisphosphonate of step (a) is administered orally.

7. The method of claim 6 wherein the bisphosphonate of step (a) is administered every day or every other day.

8. The method of claim 7 wherein the loading dose is a level of from about 3 times to about 10 times of the maintenance dose.

9. The method of claim 8 wherein the bisphosphonate of step (a) is selected from the group consisting of risedronate, alendronate acid, pamidronate, tiludonate, clodronate, cimadronate, ibandronate, zolendronate, and salts and esters thereof.

10. The method of claim 9 wherein the bisphosphonate is risedronate or alendronic acid.

11. The method of claim 4 wherein the maintenance dose is administered daily, every other day, twice a week, weekly, bi-weekly, once a month, or every other month or every three months.

12. The method of claim 11 wherein the Bisphosphonate of step (b) is selected from the group consisting of risedronate, alendronate acid, pamidronate, tiludronate, clodronate, cimadronate, ibandronate, zolendronate, and salts and esters thereof.

13. The method of claim 10 wherein the loading dose is a level of from about 3 times to about 6 times greater than the maintenance dose.

14. The method of claim 13 wherein the loading dose of risedronate is from about 15 mg to about 50 mg per day.

15. The method of claim 13 wherein the loading dose of alendronate is from about 15 mg to about 70 mg per day.

16. A kit for use in the treatment of increasing bone mass according to a regimen comprising unit doses of bisphosphonate for a loading period and unit doses of a bisphosphonate for a maintenance period which follows the loading period.

17. A kit according to claim 16 wherein the Kit further comprises means for facilitating compliance.

18. A kit according to claim 17 wherein the loading dose period is from about 7 to 180 days.

19. A kit according to claim 18 wherein the Bisphosphonate is selected from the group consisting of risedronate, alendronate acid, pamidronate, tiludronate, clodronate, cimadronate, ibandronate, zolendronate, and salts and esters thereof.

20. A kit according to claim 19 wherein the bisphosphonate is risedronate or alendronic acid.
